(19) United States
(12) Patent Application Publication
(10) Pub. No.: US 2010/0143421 A1
(43) Pub. Date: Jun. 10, 2010

(54) Delivery Device

(76) Inventors: Isabelle Van Reeth, Incourt (BE); Virginie Caprasse, Oreye (BE); Xavier Thomas, Rhode Saint-Genese (BE); Jean-Paul Lecomte, Brussels (BE); Benedicte Courel, Waterloo (BE); Esternia Schirosi, Anderlues (BE)

Correspondence Address: HOWARD & HOWARD ATTORNEYS PLLC, 450 West Fourth Street, Royal Oak, MI, 48067, US

(21) Appl. No.: 12/296,978

(22) PCT Filed: Apr. 10, 2007
(86) PCT No.: PCT/EP2007/053456

§ 371 Date: Sep. 4, 2009

(30) Foreign Application Priority Data
Apr. 13, 2006 (GB) 0607438.9

(51) Int. Cl. A61K 8/00; B01J 13/18; A61L 9/012; A61Q 13/00

(52) U.S. Cl. 424/401; 264/4.7; 424/497; 424/443; 512/4

(57) ABSTRACT

A process for the production of water-containing silicone rubber device comprising the steps of mixing an external phase comprising an organopolysiloxane polymer having at least two reactive groups per molecule, a water in oil emulsifier and where required one or more active ingredients and/or optional additives; with an internal phase comprising water and optionally one or more active ingredients to form an emulsion; and subsequently introducing a cure package comprising a cure package comprising a suitable catalyst, which will be determined by the cure reaction between the polymer and where required a cross-linker, and which contains more than two reactive groups, designed to react with the reactive groups in the polymer in the presence of said catalyst in order to cure the external phase of the composition, thereby entrapping the water within the body of said water-containing silicone rubber device. The resulting product is preferably used fragrance delivery in applications selected from aromatherapy anti stress ball, aromatherapy pillow, deodorising shoe sole solid deodorising device for houses, external prosthesis, facial mask for delivery of actives.

This invention relates to a method of preparing water-containing silicone rubber articles suitable for use as a means of controlled and substantially uniform release of one or more active ingredients. The invention further relates to the resulting articles and the controlled and substantially uniform release of active ingredients in fragrance, Cosmetic, Veterinary, pharmaceutical and/or therapeutic applications.

Oil in water emulsion of utilising condensation curable polydimethylsiloxane (PDMS) as the “oil” phase are well known. GB 093047 described the use of tin catalyst to cure PDMS polymers oil phase in an oil in water emulsion. U.S. Pat. No. 4,954,565 describes how PDMS is cured in an emulsion form using tin based catalysts. Two part PDMS systems in the form of oil in water emulsions are described in U.S. Pat. No. 4,590,220 and U.S. Pat. No. 391,899. They react upon mixing. Such systems typically result in fast reactions occurring during the cure process resulting in the production of open-cell, elastomeric foam by forming a froth from the emulsion and removing the water from the froth. All the above emulsions contain water as the external phase, and the PDMS in the emulsion can only become solid after the removal of the water resulting in anhydrous cured silicone rubber products.

U.S. Pat. No. 6,379,689 describes an article which comprises an elastomeric silicone matrix in which an active organic material, and a “compatibilizer” are dispersed. The compatibilizer is identified as “the essential constituent” and is defined as an organic solvent having a Hildebrandt solubility parameter of from 8 to 14 (cal/cm³)^{1/2} and a vapour pressure of from 0.06 to 105 Pa at 20° C. Resulting cured products are used for the controlled liberation of active volatile organic materials e.g. a perfume, insecticide or insect repellent into the atmosphere. U.S. Pat. No. 6,379,689 teaches that an emulsifier can be introduced into the composition as a means of avoiding the sweating or exudation of volatile active ingredients from the silicone rubber matrix during storage. No water is incorporated into this system.

WO 03/101404 describes topical preparations for release of an active agent and methods of making and using such topical preparations. The preparations have an internal phase, typically in the form of a hydrophilic carrier and an active agent, dispersed within an external phase which is typically a silicone matrix. WO 03/101404 describes the use of different silicone matrices such as high molecular weight polydimethylsiloxanes, loosely or lightly cross-linked silicone elastomers, fillerless elastomers, cellular elastomers, silicone rubbers, silicone pressure sensitive adhesives, and combinations thereof together with the use of a silicone based surfactant. The process for preparing the topical preparation all describe the use of a one part silicone composition as the external phase into which the internal phase is introduced and the silicone component is subsequently cured. Whilst water is listed as a possible constituent of the internal phase, generally most examples rely on alcohols and glycols as bulk constituents of the internal phase. The resulting uncured product is case into a film prior to application to the skin or is applied directly to the skin where it cures in situ. This system using reticulating silicones lacks flexibility with respect to both the type of additives used and the process conditions.

Although WO 03/101404 describe a topical preparation which at best results in a film, for release of an active, there is still a need to provide a soft to hard silicone rubber device with original texture and feel (wet or oily) containing up to 35% of water phase and or up to 20% of fragrances to be used in topical and non topical applications in Personal Care, Household and Health care applications. There is also a need for a silicone rubber with broad formulation flexibility both on the external phase and internal phase in order to achieve a large choice of texture, feels and applications. A person skilled in the art would expect the desired size of the device, if containing the amount of water desired, to result in a very fragile article which would fall apart under the least stress.

Surprisingly the inventors have found that stable devices can be obtained with very large water contents which may be utilised for a wide variety of applications as will be described hereafter.

In accordance with a first aspect of the present invention there is provided a process for the production of water-containing silicone rubber device comprising the steps of mixing
I. an external phase comprising an organopolysiloxane polymer having at least two reactive groups per molecule, a water in oil emulsifier and where required one or more active ingredients and/or optional additives; with
II. an internal phase comprising water and optionally one or more active ingredients to form an emulsion; and subsequently introducing a cure package comprising
III. a cure package comprising a suitable catalyst, which will be determined by the cure reaction between (i) and where required a cross-linker, and which contains more than two reactive groups, designed to react with the reactive groups in polymer (i) in the presence of said catalyst in order to cure the external phase of the composition, thereby entrapping component (ii) within the body of said water-containing silicone rubber device.

In accordance with a second aspect of the present invention there is provided a water-containing silicone rubber device obtainable by the following method comprising the steps of mixing
(i) an external phase comprising a organopolysiloxane polymer having at least two reactive groups per molecule, a water in oil emulsifier and where required one or more active ingredients and/or other optional additives; with
(ii) an internal phase comprising water and optionally one or more active ingredients to form an emulsion; and subsequently introducing a cure package comprising
(iii) a cure package comprising a catalyst, which will be determined by the cure reaction between (i) and where required a cross-linker, and which contains more than two reactive groups, designed to react with the reactive groups in polymer (i) in the presence of said catalyst in order to cure the external phase of the composition, thereby entrapping component (ii) within the body of said water-containing silicone rubber device.

More specifically the invention relates to a device for e.g. controlled and substantially uniform release of volatile organic substances into the surrounding atmosphere, having a three dimensional shape, where each dimension has a minimum size of at least 1 millimetre, the device comprising a silicone elastomer having water dispersed in it to an extent of up to and preferably at least 35% by weight based on the device. The composition is made using a 2 part silicone elastomer system and a water-in-silicone emulsifier and water as discussed above. The water containing silicone rubber can have a definite shape depending of the mould used or can be in the form of a thin sheet.

Despite the teaching of U.S. Pat. No. 6,379,689 the inventor's have found that no “compatibilizer” as defined above is required in the composition of the present invention.

Preferably the composition in accordance with the invention is cured by way of a condensation reaction in which case the reactive groups on the organopolysiloxane polymer having at least two reactive groups per molecule polymer in the external phase are —OH groups and/or condensable groups and the cure package comprises a suitable condensation catalyst and a suitable cross-linker. Alternatively the composition may be cured using a suitable free radical polymerisation process.

When the composition is cured via a condensation reaction system the polymer in the external layer (i) comprises at least two condensable groups preferably terminal groups, that will, in appropriate conditions, undergo a condensation reaction. Preferably the condensable groups in the present invention are hydroxyl or hydrolysable end groups (e.g. alkoxy groups). Hence, the polymer has the general formula

X-A-X³ (1)

X and X³ are independently selected from siloxane groups which terminate in hydroxyl or hydrolysable groups and A is a siloxane containing polymeric chain.
Examples of hydroxyl-terminating or hydrolysable groups which terminate X or X³ include —Si(OH)₃, (R^{a})Si(OH)₂, —(R^{a})₂SiOH, —R^{a}Si(OR^{b})₂, —Si(OR^{b})₃, —R^{a}₂SiOR^{b} or —R^{a}₂Si—R^{c}—SiR^{d}ₙ(OR)₃₋ₙ where each R^{a} independently represents a monovalent hydrocarbyl group, for example, an alkyl group, in particular having from 1 to 8 carbon atoms, (and is preferably methyl); each R^{b} and R^{d} group is independently an alkyl or alkoxy group in which the alkyl groups suitably have up to 6 carbon atoms; R^{c} is a divalent hydrocarbon group which may be interrupted by one or more siloxane spacers having up to six silicon atoms; and n has the value 0, 1 or 2. Preferably X and/or X³ contain hydroxyl groups or groups which are otherwise hydrolysable in the presence of moisture. Preferably however the reactive polymer in the external phase of the present embodiment is a substantially linear diorganopolysiloxanes containing two, or approximately two, terminal silanol (—SiOH) groups per molecule. Some of the silanol groups can be substituted by trimethylsilyl groups, when lower cross-linking density is desired.

The silicon-bonded groups present in the backbone of the reactive diorganopolysiloxane polymer of the external phase (i) are selected from monovalent hydrocarbon groups, preferably having 1 to 12 carbon atoms, monovalent halogenated hydrocarbon groups, preferably having 1 to 12 carbon atoms and cyanoalkyl radicals. Examples of the organic radicals which may be present are alkyl radicals such as methyl, ethyl, propyl, isopropyl, butyl, tertiary butyl, dodecyl or octadecyl radicals, alkenyl radicals such as vinyl, allyl hexenyl and cyclohexenyl, aryl radicals such as phenyl and naphthyl and halogenated hydrocarbon radicals such as chlorophenyl, bromomethyl and trifluoropropyl radicals.

The hydroxyl terminated diorganopolysiloxanes of component (i) may vary in viscosity within the range from 50 to 60,000 mPa·s at 25° C. Most preferred hydroxyl terminated diorganopolysiloxanes are of the formula HO-[-alkyl₂SiO—]ₘ—H particularly those where each alkyl radical is a methyl group and m has a value such that the viscosity of the diorganopolysiloxane lies within the range from 500 to 60,000 mPa·s at 25° C.

When the composition of the present invention is cured by way of a condensation reaction process the cure package comprises a suitable condensation catalyst and a suitable cross-linker.

Any suitable condensation catalyst may be utilised as the catalyst of the cure package. Such materials and their use as catalysts in two-package cold-curable systems are well-known and the choice of the most suitable catalysts for a particular formulation or application will be readily apparent to those skilled in the art. The preferred catalysts are the metal salts of carboxylic acids, for example zinc naphthenate, lead octoate, stannous acetate, dialkyltin esters including dimethyldineodecanoate, dibutyltin dilaurate, dibutyltin diacetate and dioctyltin diacetate, the most preferred of these being the organo tin salts, and more specifically dialkyl tin salts. A mixture of more than one type of condensation catalyst may be present the cure package if/when preferred. A sufficient amount of the respective catalyst to cure the composition will be used. Preferably from 0.2% to 2% by weight of the total composition of the tin based catalysts may be used in a composition of the present invention.

Titanate and/or zirconate based condensation catalysts may alternatively be used as the condensation catalyst of the cure package. These may comprise a compound according to the general formula Ti[OR⁵]₄ and Zr[OR⁵]₄ respectively where each R⁵ may be the same or different and represents a monovalent, primary, secondary or tertiary aliphatic hydrocarbon group which may be linear or branched containing from 1 to 10 carbon atoms. Optionally the titanate may contain partially unsaturated groups. However, preferred examples of R⁵ include but are not restricted to methyl, ethyl, propyl, isopropyl, butyl, tertiary butyl and a branched secondary alkyl group such as 2,4-dimethyl-3-pentyl. Preferably, when each R⁵ is the same, R⁵ is an isopropyl, branched secondary alkyl group or a tertiary alkyl group, in particular, tertiary butyl.

Alternatively, the titanate may be chelated. The chelation may be with any suitable chelating agent such as an alkyl acetylacetonate such as methyl or ethylacetylacetonate. Any suitable chelated titanates or zirconates may be utilised. Preferably the chelate group used is a monoketoester such as acetylacetonate and alkylacetoacetonate giving chelated titanates such as, for example diisopropyl bis(acetylacetonyl)titanate, diisopropyl bis(ethylacetoacetonyl)titanate, diisopropoxytitanium Bis(Ethylacetoacetate) and the like. Examples of suitable catalysts are additionally described in EP1254192 and WO200149774 which are incorporated herein by reference.

The amount of titanate and/or zirconate Catalyst would be from 0.1% to 3.0%, preferably from 0.3 to 1.0% and more preferably from 0.4% to 0.6% by weight of the composition.

For condensation type cure packages a cross-linker is additionally required as part of the cure package. Any suitable cross-linker may be utilised. Such a cross-linker may be selected from one or more silanes or short chain siloxane each of which silane and short chain siloxane contain 3 or more groups which will undergo a condensation reaction with the hydroxyl and/or hydrolysable groups of the reactive polymer in the external phase. Preferably each silane cross-linker in the cure package contains 3 or 4 alkoxy, acetoxy and/or oximo groups, most preferably the reactive groups are alkoxy groups having at least three alkoxy groups. Preferably each of the alkoxy, acetoxy and oximo groups comprise from 1 to 10 carbon atoms, most preferably from 1 to 6 carbon atoms. Preferably the groups present in the silane not adapted to react with the hydroxyl or hydrolysable groups of the reactive polymer in the external phase are aryl or alkyl groups, preferably said alkyl groups comprise from 1 to 10 carbon atoms, most preferably from 1 to 6 carbon atoms. Alternatively but not preferred these groups which are non-reactive with hydroxyl groups may be alkenyl groups. Silanes which can be used as cross-linkers include alkyltrialkoxysilanes such as methyltrimethoxysilane (MTM) and methyltriethoxysilane, alkenyltrialkoxy silanes such as vinyltrimethoxysilane and vinyltriethoxysilane, isobutyltrimethoxysilane (iBTM). Other suitable silanes include ethyltrimethoxysilane, vinyltriethoxysilane, phenyltrimethoxysilane, alkoxytrioximosilane, alkenyltrioximosilane, 3,3,3-trifluoropropyltrimethoxysilane, methyltriacetoxysilane, vinyltriacetoxysilane, ethyl triacetoxysilane, dialkoxydiacetoxysilane, phenyl-tripropionoxysilane, methyltris(methylethylketoximo)silane, vinyl-tris-methylethylketoximo)silane, methyltris(methylethylketoximino)silane, methyltris(isopropenoxy)silane, vinyltris(isopropenoxy)silane. Exemplary tetralkoxysilanes include tetrapropoxysilane and tetrabutoxysilane. The amount of the silane-based cross linker would be from 0.0% to 10.0%, preferably from 2.0% to 5.0% and more preferably from 2.5% to 4.0% by weight of the composition.

Preferred trialkoxy-functionalised siloxanes for use as cross-linkers in the cure package are polydiorganosiloxanes end-blocked with alkoxysilethylene groups at both ends of the polymer and are well known in the art, for example, as described in U.S. Pat. No. 3,175,993, U.S. Pat. No. 4,772,675 and U.S. Pat. No. 4,962,174 said siloxanes are incorporated herein by reference. Polydiorganosiloxanes partially end-blocked with substituted silethylene groups, as described in U.S. Pat. No. 6,037,434 (which siloxanes are incorporated herein by reference) may be used as well, when lower cross-linking density is desired. The amount of trialkoxy-functionalised siloxane used as cross-linker will be calculated prior to use and may for example be determined on the basis of a stoichiometric equivalence between hydroxyl groups from the reactive polymer in the external phase and alkoxy groups from the cross-linker of the cure package. The use of trialkloxy-functionalised siloxane enable the making of formulation without the use of silanes.

Whilst compositions in accordance with the present invention are preferably cured by condensation reactions, in some circumstances free radical polymerisation processes, specifically those using innovative organoboron amine catalyst complexes as described in WO2006/073696 may be utilised in vulcanisable compositions in accordance with the present invention such that the siloxane polymer in the external phase in accordance with the present invention comprises a free radical polymerisable organosiloxane monomer, oligomer or polymer; and the cure package comprises an organoboron amine catalyst complex and optionally an amine reactive compound having amine reactive groups may be added to the external phase, internal phase or cure package. Alternatively using this cure process the catalyst may be at least partially added into the external phase and the amine reactive compound may be incorporated in the internal phase, or may be introduced as the cure package after the combination of the internal and external phases.

In such a composition the reactive polymer in the external phase of the present invention may be an organopolysiloxane having linear, branched, hyperbranched, or resinous structures. The compound can be homopolymeric or copolymeric. The free radical polymerisable moiety for such an organopolysiloxane can be an unsaturated organic group such as an alkenyl group having 2-12 carbon atoms exemplified by vinyl, allyl, butenyl, and hexenyl groups. The unsaturated organic groups can also consist of alkynyl groups having 2-12 carbon atoms exemplified by ethynyl, propynyl, and butynyl groups. The unsaturated organic group can bear the free radical polymerisable group on oligomeric or polymeric polyethers such as allyloxypoly(oxyalkylene) groups and halogen substituted analogs thereof. The free radical polymerisable organic groups can contain acrylate or methacrylate functional groups exemplified by acryloxyalkyl groups such as 3-acryloxypropyl, 2-acryloxyethyl, acryloxymethyl, and methacryloxyalkyl groups such as 3-methacryloxypropyl, 2-methacryloxyethyl, and methacryloxymethyl. The unsaturated organic groups can be located at terminal positions, pendant positions, or both terminal and pendant positions relative to the polymer backbone. The preferred free radical polymerisable moiety for monomeric, oligomeric, and polymeric organosilicon compounds are acrylate and methacrylate groups.

Any remaining silicon bonded organic groups can be monovalent organic groups free of aliphatic unsaturation. The monovalent organic group can have 1-20 carbon atoms, preferably 1-10 carbon atoms, and is exemplified by alkyl groups such as methyl, ethyl, propyl, pentyl, octyl, undecyl, and octadecyl; cycloalkyl groups such as cyclohexyl; aryl groups such as phenyl, tolyl, xylyl, benzyl, and 2-phenylethyl; alkyloxypoly(oxylalkylene) groups such as propyloxypoly(oxyethylene), propyloxypoly(oxypropylene), propyloxy-poly(oxypropylene)-co-poly(oxyethylene) groups, halogen substituted versions thereof; cyanofunctional groups such as cyanoalkyl groups exemplified by cyanoethyl and cyanopropyl; carbazole groups such as 3-(N-carbazolyl)propyl; arylamino-functional groups such as 4-(N,N-diphenylamino)phenyl-3-propyl; and halogenated hydrocarbon groups such as 3,3,3-trifluoropropyl, 3-chloropropyl, dichlorophenyl, and 6,6,6,5,5,4,4,3,3-nonafluorohexyl.

The reactive polymer in accordance with the external phase of the present invention may be a fluid having a viscosity of 0.001 Pa·s at 25° C. to a compound having the consistency of a gum.

The organoborane amine complex is a complex formed between an organoborane, and a suitable amine compound that renders the complex stable at ambient conditions. The complex should be capable of initiating polymerization or cross-linking of reactive polymer in accordance with the external phase of the present invention by the introduction of an amine reactive compound and/or by heating. An example is an alkylborane amine complex formed from trialkylboranes and various amine compounds. Examples of trialkylboranes useful for forming the organoborane amine complex include trialkylboranes of the formula BR″₃ where R″ represents linear and branched aliphatic or aromatic hydrocarbon groups containing 1-20 carbon atoms. Some examples include triethylborane, tri-n-butylborane, tri-n-octylborane, tri-sec-butylborane, tridodecylborane, and phenyldiethylborane.

Some examples of amine compounds useful to form the organoborane amine complex with the organoborane compounds include 1,3 propane diamine, 1,6-hexanediamine, methoxypropylamine, pyridine, and isophorone diamine. Other examples of amine compounds useful to form organoborane amine complexes are described in U.S. Pat. No. 6,806,330.

Silicon containing amine compounds can also be used to form the organoborane amine complex including compositions such as aminomethyltrimethoxysilane, 3-aminopropyltrimethoxysilane, aminomethyltriethoxysilane, 3-aminopropyltriethoxysilane, 2-(trimethoxysilylethyl)pyridine, aminopropylsilanetriol, 3-(m-aminophenoxy)propyltrimethoxysilane, 3-aminopropyldiisopropylmethoxysilane, aminophenyltrimethoxysilane, 3-aminopropyltris(methoxyethoxy)silane, N-(2-aminoethyl)aminomethyltrimethoxysilane, N-(2-aminoethyl)-3-aminopropyltrimethoxysilane, N-(6-aminohexyl)aminomethyltrimethoxysilane, N-(2-aminoethyl)-11-aminoundecyltrimethoxysilane, (aminoethylaminomethyl)phenethyltrimethoxysilane, N-(2-aminoethyl)-3-aminopropylmethyldimethoxysilane, N-(2-aminoethyl)-3-aminoisobutylmethyldimethoxysilane, (3-trimethoxysilylpropyl)diethylene-triamine, and 1,1,2,4-tetramethyl-1-sila-2-azacyclopentane.

Amine functional organopolysiloxanes are also useful for forming the organoborane amine complex including organopolysiloxane resins. This is subject to the stipulation that the molecule contain at least one amine functional group, such as aminomethyl, 2-aminoethyl, 3-aminopropyl, 6-aminohexyl, 11-aminoundecyl, 3-(N-allylamino)propyl, N-(2-aminoethyl)-3-aminopropyl, N-(2-aminoethyl)-3-aminoisobutyl, p-aminophenyl, 2-ethylpyridine, and 3-propylpyrrole.

Specific examples include terminal and/or pendant amine-functional polydimethylsiloxane oligomers and polymers, terminal and/or pendant amine-functional random, graft and block copolymers and co-oligomers of polydimethylsiloxane and poly(3,3,3 trifluoropropyl-methylsiloxane), terminal and/or pendant amine-functional random, graft and block copolymers and co-oligomers of polydimethylsiloxane and poly(6,6,6,5,5,4,4,3,3-nonfluorohexyl-methylsiloxane), and terminal and/or pendant amine-functional random, graft and block copolymers and co-oligomers of polydimethylsiloxane and polyphenymethylsiloxane. Other examples of useful compounds include resinous amine-functional siloxanes such as the amine-functional compounds described previously as organopolysiloxane resins.

Also useful to form the organoborane amine complex are other nitrogen containing compounds including N-(3-triethyoxysilylpropyl)-4,5-dihydroimidazole, ureidopropyltriethoxysilane, siloxanes of formulas similar to formulas (a) and (b) noted above, and those compounds described previously as organopolysiloxane resins in which at least one group is an imidazole, amidine, or ureido functional group. When the amine compound is polymeric, the molecular weight is not limited, except that it should be such as to maintain a sufficiently high concentration of boron to permit curing or polymerization of the composition. For example, in a two-part composition, the part containing the organoborane initiator may be diluted with other components of the composition, or it may consist of the initiator complex alone.

If desired, the curable composition may be stabilized by physically or chemically attaching the organoborane amine complex to solid particles. This provides a way to control working times, as well as to stabilize liquid phase organoborane amine complexes against separating from the rest of the composition during storage. For example, chemical attachment can be performed by pretreating solid particles such as ground silica, precipitated silica, calcium carbonate, or barium sulphate, with a condensation reactive compound containing an amine group such as aminopropyltrimethoxysilane. The pretreatment is followed by complexation with an organoborane compound, or by the direct treatment of the solid particles using a preformed organoborane amine complex that is condensation reactive. When the solid particles contain surface functional groups, additives such as surface treating agents or impurities that are inherently amine reactive, require appropriate pre-cautions to avoid premature decomplexation of the organoborane amine complex being attached. Solid particles containing amine reactive substances can be purified or neutralized before attachment of the organoborane amine complex. Alternatively, the attachment of the organoborane amine complex can be performed in an oxygen free environment.

The curable composition may contain an amine reactive compound that is capable of initiating the polymerization, or cross-linking, when mixed with the organoborane amine complex and exposed to an oxygenated environment. The amine reactive compound may be a liquid, gas, or solid. The amine reactive compound may be a small molecule, a monomer, an oligomer, a polymer, or a mixture thereof, may also be diluted or borne by a carrier such as an aqueous or non-aqueous solvent, or by a filler particle. The amine reactive compound may contain free radical polymerisable groups or other functional groups such as a hydrolyzable group. The amine reactive groups on the amine reactive compound may be borne on an organic, organosilicon, or organopolysiloxane compound. The presence of the amine reactive compound allows the initiation of polymerization or cross-linking to occur at temperatures below the dissociation temperature of the organoborane amine complex including room temperature and below.

To achieve storage stability in the presence of oxygen, it is preferred that the organoborane amine complex and the amine reactive compound (when present) be physically or chemically isolated from each other. For example, a composition containing the amine reactive compound can be rendered air stable by packaging it separately from the organoborane amine complex as a multi-component formulation. Alternatively, the organoborane amine complex and the amine reactive compound or both can be encapsulated, or delivered in separate phases. This can be accomplished by introducing one or both of the organoborane amine complex and the amine reactive compound in a solid form that prevents intimate mixing of the organoborane amine complex and the amine reactive compound Curing of the composition can be activated by (a) heating it above the softening temperature of the solid phase component or encapsulant, or (b) by introduction of a solubilising agent that allows mixing of the organoborane amine complex and the amine reactive compound. The organoborane amine complex and the amine reactive compound can also be combined in a single container without significant polymerization or cross-linking by packaging the two components in a container where mixing conditions are anaerobic.

Examples of some amine reactive compounds having amine reactive groups that can rapidly initiate polymerization or cure in the presence of oxygen include mineral acids, Lewis acids, carboxylic acids, carboxylic acid derivatives such as anhydrides and succinates, carboxylic acid metal salts, isocyanates, aldehydes, epoxides, acid chlorides, and sulphonyl chlorides. Some suitable amine reactive compounds include acrylic acid, methacrylic acid, polyacrylic acid, polymethacrylic acid, methacrylic anhydride, undecylenic acid, oleic acid, lauric acid, lauric anhydride, citraconic anhydride, ascorbic acid (Vitamin C), methylene bis-(4-cyclohexylisocyanate) monomers or oligomers, hexamethylene diisocyanate monomers or oligomers, toluene-2,4-diisocyanate monomers or oligomers, isophorone diisocyanate monomers or oligomers, methylene diphenyl isocyanate monomers or oligomers, methacryloylisocyanate, 2-(methacryloyloxy)ethyl acetoacetate, undecylenic aldehyde, and dodecyl succinic anhydride.

For improved compatibility in curable compositions herein containing organosiloxane matrices, it may be advantageous when the amine reactive compound is an organosilane or organopolysiloxane bearing amine reactive groups. Some examples include isocyanatomethyltrimethoxysilane; 3-isocyanatopropyltrimethoxysilane; 3-glycidoxypropyltrimethoxysilane; triethoxysilylpropyl succinic anhydride; propylsuccinic anhydride functionalized linear, branched, resinous, and hyperbranched organopolysiloxanes; methylsuccinic anhydride functionalized linear, branched, resinous, and hyperbranched organopolysiloxanes; cyclohexenyl anhydride functional linear, resinous, and hyperbranched organopolysiloxanes; carboxylic acid functionalized linear, branched, resinous, and hyperbranched organopolysiloxanes such as carboxydecyl terminated oligomeric or polymeric polydimethylsiloxanes; and aldehyde functionalized linear, branched, resinous, and hyperbranched organopolysiloxanes such as undecylenic aldehyde-terminated oligomeric or polymeric polydimethylsiloxanes.

This vulcanisable/curable composition is used in amounts of from 10 to 50 parts by weight, preferably from 20 to 40 parts by weight, more preferably from 25 to 35 parts by weight of the total weight of the composition.

Irrespective of the reactive polymer of the external phase and the cure package, the external phase additionally comprises a water in oil (w/o) emulsifier, preferably a silicone w/o emulsifier and even more preferably a water-in-silicone emulsifier is preferably utilised in the composition in accordance with the present invention. Such an emulsifier typically has a hydrophilic lipophilic balance of less than 5 (HLB>5). Typically these are water insoluble. Preferably said water-in-silicone emulsifier is non-ionic. Preferably the water-in-silicone emulsifier is selected from the group comprising polyoxyalkylene-substituted silicones, silicone alkanolamides, silicone esters and silicone glycosides. Suitable silicone-based emulsifiers (sometimes referred to as surfactants) are well known in the art, and have been described for example in U.S. Pat. No. 4,122,029, U.S. Pat. No. 5,387,417, and U.S. Pat. No. 5,811,487 and include polydiorganosiloxane polyoxalkylene copolymers containing at least one polydiorganosiloxane segment consisting essentially of a plurality of siloxane units having the general formula:—

R_{b}SiO_{(4-b)}/2

wherein b has a value of 0 or an integer of from 1 to 3. Preferably the average value of b for the total number of siloxane units per copolymer molecule is about 2, and R denotes a radical selected from the group consisting of methyl, ethyl, vinyl, phenyl, and a divalent radical bonding a polyoxyalkylene segment to the polydiorganosiloxane segment. Preferably at least 95% of all R groups are methyl. Preferably each copolymer comprise at least one polyoxyalkylene segment having an average molecular weight of at least 1000 and consisting of from 0 to 50 mol percent polyoxypropylene units and from 50 to 100 mol percent polyoxyethylene units. At least one terminal portion of said polyoxyalkylene segment is bonded to said polydiorganosiloxane segment. Any terminal portion of said polyoxyalkylene segment not bonded to said polydiorganosiloxane segment comprises a terminating radical. Preferably the weight ratio of polydiorganosiloxane segments to polyoxyalkylene segments in said copolymer having a value of from 2:1 to 8:1.

Alternatively the silicone-based surfactant may be a cross-linked emulsifier in which at least two organopolysiloxane-polyoxyalkylene molecules are cross-linked by a cross-linking radical; the cross-linked organopolysiloxane-polyoxyalkylene emulsifier having the formula:—

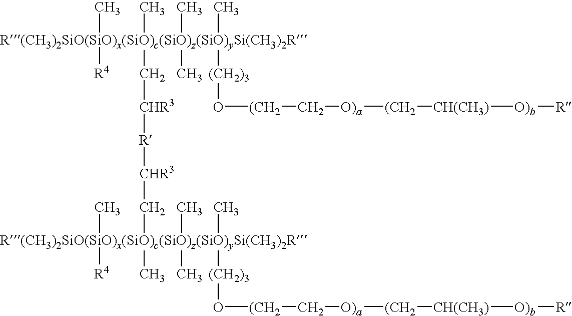

wherein R⁴ is an aliphatic radical having 2 to 25 carbon atoms; R′ is an organic or organosiloxane group which does not contain hydrolysable bonds; R″ is a terminal group; each R′″ is independently an aliphatic radical having 1 to 25 carbon atoms; R³ is independently selected from the group consisting of hydrogen and an aliphatic radical containing 1-3 carbon atoms; x is zero or an integer from 1 to 100; c is an integer from 1 to 5; z is 0 or an integer from 1 to 600; y is an integer from 1 to 10; x+y+z>40; a is an integer from 4 to 40; b is 0 or an integer from 1 to 40; a/b>1.

Examples of commercial water-in-silicone emulsifiers are Dow Corning® 5225c Formulation Aid, Dow Corning® 3225c Formulation Aid Dow Corning® 5200c Formulation Aid, BY-11030, DC 9011, all from Dow Corning Corporation, Abil EM 90, EM 97 (Degussa), SF 1540 (General Electric), KF 6017, 6038, KSG 210, 320 (Shinetsu). ABn silicone polyether can also be comprised into the composition such as Dow Corning® FZ-2231, Dow Corning® FZ-2233. The amount of the silicone emulsifying agent in the final composition may vary widely, but typically would be from 0.05% to 10%, preferably 0.1 to 5%, more preferably 0.15 to 4.0 by weight, most preferably 0.2 to 3.0% by weight.

The internal phase of the present invention comprises water preferably at least 20% by weight of the composition. The uncured composition of the present invention comprises an internal phase in the form of an aqueous component. For purposes of this invention the term “aqueous component” refers to any material consisting essentially of, or predominantly of, water. Generally the internal phase comprises water or water and an active ingredient and optionally additional suitable additives (e.g. electrolytes). The internal phase is present in an amount of from about 10% to 60%, preferably from about 15% to 50% and more preferably from 20% to 40% by weight of the total uncured composition.

The internal phase (component (ii)) may also comprise one or more additives including, for example one or more electrolytes may be present in the water phase of the uncured composition of the present invention. Preferred electrolytes are alkali metal salts and alkaline earth salts, especially the chloride, borate, citrate, and sulphate salts of sodium, potassium, calcium and magnesium. When present these electrolytes are present in amounts typically from 0.5 to about 3 wt % and more preferably from 1.0 to 2.0 wt % of the total composition.

Preferably active ingredients when introduced into the composition in the external phase are either:—
(i) in a liquid form which is soluble or miscible with the polymer in the external phase or
(ii) in solid form is soluble in the external phase of the composition in accordance with the present invention.

Preferably active ingredients when introduced into the composition in the internal phase are either:—
(iii) in a liquid form which is soluble or miscible with water or
(iv) in solid form is soluble in water such the internal phase of the emulsion is an aqueous solution.

In accordance with the present invention any suitable active ingredient may be incorporated into the internal phase. Preferably these active ingredients are provided in liquid form and most preferably comprise fragrances, essential oils and the like suitable for controlled release from a cured product resulting from the composition in accordance with the present invention. In the case of the present invention one of the main functions of the cured product is as a means of gradually releasing a fragrance and/or perfume over an extended period of time. Any suitable perfume and/or fragrance commonly used in the perfume industry may be utilised for this purpose. These perfumes and/or fragrances typically belong to a variety of chemical classes, as varied as alcohols, aldehydes, ketones, esters, ethers, acetates, nitrites, terpenic hydrocarbons, heterocyclic nitrogen or sulphur containing compounds, as well as essential oils of natural or synthetic origin. Many of these perfume ingredients are described in detail in standard textbook references such as Perfume and Flavour Chemicals, 1969, S. Arctander, Montclair, N.J. The amount of perfume and/or fragrance when present may be as much as 30% by weight %, more preferably from 1.0% to 20.0% and more preferably from 2% to 20.0% by weight of the composition.

However, whilst the above is currently seen as the main use for such a product it is envisaged that products of the present invention may additionally or alternatively be utilised in Cosmetic, Veterinary, pharmaceutical and therapeutic applications. For the latter applications the active ingredients may include preservatives, vitamins and their derivatives, whitening agents, anti-oxidants, ceramides, amino-acid derivatives, polyols, such as glycerine and propylene glycol providing said polyols are used in addition to water and do not replace water, and botanicals (plant extracts) conditioning agents for skin.

Other active ingredients may include, depending on the use, sunscreen agents, humectants, emollients, occlusive agents, and esters, anti acne agents, antimicrobial agents, antiperspirant agents and deodorant agents, cosmetic biocides, oxidizing agents, reducing agents, skin bleaching agents, skin protectants, cleansing agents such as anionic detersive surfactant, foam boosting agents, insect repellents, agents for artificially tanning and/or browning the skin (self-tanning agents), such as, for example, dihydroxyacetone (DHA), pH control agents, pharmaceutical actives. For pharmaceutical and therapeutic agents, they are exemplified by anti-acne agent, antibiotic, antiseptic, antifungal, antibacterial, antimicrobial, biocides, anti-inflammatory, astringents, hormones, anticancer agents, smoking cessation compositions, cardiovascular, histamine blocker, bronchodilator, analgesic, anti-arrythmic, antihistamine, alpha-I blocker, beta blocker, ACE inhibitor, diuretic, anti-aggregant, sedative, tranquilllizer, anticonvulsant, anticoagulant agents, vitamins, anti-aging agents, agents for treating gastric and duodenal ulcers, anti-cellulites, proteolytic enzymes, healing factors, cell growth nutrients, peptides and others. Specific examples of suitable therapeutic active agents include penicillins, cephalosporins, tetracyclines, macrolides, epinephrine, amphetamines, aspirin, acetominophen, barbiturates, catecholamines, benzodiazepine, thiopental, codeine, morphine, procaine, lidocaine, benzocaine, sulphonamides, ticonazole, perbuterol, furosamide, prazosin, prostaglandins, salbutamol, indomethicane, diclofenac, glafenine, dipyridamole, theophylline and retinol.

Whilst it is possible to have different active ingredients introduced into the same composition using both the external and internal phases, preferably all active ingredients are introduced into the composition in one of the internal or external phase per composition.

A broad range of different silicone based unreactive materials may be introduced into the composition of the present invention. By unreactive it is meant that such compounds do not participate in the cure process. These compounds function as diluents to ensure the emulsification of the internal and external layers and upon cure to provide the cured product with a variety of textures and feel.

The unreactive silicones may include, for example:—

non-volatile polysiloxanes of the structure:

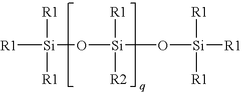

wherein q has a value sufficient to provide polysiloxane polymers having a viscosity in the range of from 100 to 10,000 mPa·s. at 25° C., R1 and R2 can be alkyl radicals containing 1-20 carbon atoms or aryl groups, preferably alkyl radicals containing 1-6 carbon atoms, and more preferably methyl or phenyl groups. Typically, the value of q is from 20 to 500, more preferably from 80 to 375. Some illustrative polysiloxane polymers include trimethylsilyl terminated polydimethylsiloxane, triethylsilypolydiethylsiloxane, trialkylsilyl terminated polymethylethylsiloxane, trialkylsilyl terminated polymethylphenylsiloxane, and trialkyl or triphenylsilyl terminated polydiphenylsiloxane.

Alkylmethylsiloxanes: These siloxane polymers generally will have the formula Me₃SiO[Me₂SiO]_{y}[MeRSiO]_{z}SiMe₃, in which R is a hydrocarbon group containing 6-30 carbon atoms, Me represents methyl, and the degree of polymerization (DP), i.e., the sum of y and z is 3-50. Both the volatile and liquid species of alkylmethysiloxanes can be used in the composition.

Low molecular weight silicone such as volatile polydimethylsiloxanes such as hexamethyldisiloxane, octamethyltrisiloxane, Octamethyltrisiloxane and Dimethicone and cyclic siloxanes comprising from 1 to 15 silicon atoms such as decamethylcyclopentasiloxane. All such low molecular weight siloxanes will have a viscosity <5 mPa·s at 25° C. may be utilised in a composition in accordance with the present invention. When present the amount of the low molecular weight volatile polydimethylsiloxane in the final composition may vary widely, but typically would be from 10% to 50%, preferably 15 to 45%, more preferably 19 to 39% by weight.

Silicone gums: Polydiorganosiloxane gums are known in the art and are available commercially. They consist of generally insoluble polydiorganosiloxanes having a viscosity in excess of 1,000,000 mPa·s at 25° C., preferably greater than 5,000,000 mPa·s at 25° C. These silicone gums are typically sold as compositions already dispersed in a suitable solvent to facilitate their handling. Ultra-high viscosity silicones can also be included as optional ingredients. These ultra-high viscosity silicones typically have a kinematic viscosity greater than 5 million mPa·s at 25° C., to about 20 million mPa·s at 25° C. Compositions of this type in the form of suspensions are most preferred, and are described for example in U.S. Pat. No. 6,013,682.

Silicone polyamides: Representative compositions of suitable silicone polyamide copolymers are set forth in detail in U.S. Pat. No. 5,981,680.

Silicone resins. These resin compositions are generally highly cross-linked polymeric siloxanes. Comprising units of the general formula siloxane units having the general formula:—

R_{b}SiO_{(4-b)}/2

wherein R is as hereinbefore described, b has a value of 0 or is an integer of from 1 to 3. Cross-linking occurs due to a high proportion (>50%) of the units having a value for b of 0 or 1. In general, any silicone having a sufficient level of trifunctional and tetrafunctional siloxane monomer units, and hence possessing sufficient levels of cross-linking to dry down to a rigid or a hard film can be considered to be suitable for use as the silicone resin. Commercially available silicone resins suitable for applications herein are generally supplied in an unhardened form in low viscosity volatile or non-volatile silicone fluids. The silicone resins should be incorporated into compositions of the invention in their non-hardened forms rather than as hardened resinous structures.

Silicone elastomers: Such elastomers are generally reaction products obtained by combining an organopolysiloxane having at least two unsaturated group bound to a terminal silicon atom and an organohydrogensiloxane, and then subjecting it to at least a partial cure via a hydrosilylation reaction pathway in the presence of a suitable catalyst. Such hydrosilylation catalysts are generally chosen from platinum group metal-containing catalysts. By “platinum group metal” it is meant ruthenium, rhodium, palladium, osmium, iridium, and platinum.

Hydrosilylation catalysts are illustrated by the following; chloroplatinic acid, alcohol modified chloroplatinic acids, olefin complexes of chloroplatinic acid, complexes of chloroplatinic acid and divinyltetramethyldisiloxane, fine platinum particles adsorbed on carbon carriers, platinum supported on metal oxide carriers such as Pt(Al₂O₃), platinum black, platinum acetylacetonate, platinum(divinyltetramethyldisiloxane), platinous halides exemplified by PtCl₂, PtCl₄, Pt(CN)₂, complexes of platinous halides with unsaturated compounds exemplified by ethylene, propylene, and organovinylsiloxanes, styrene hexamethyldiplatinum, Such noble metal catalysts are described in U.S. Pat. No. 3,923,705, incorporated herein by reference to show platinum catalysts. One preferred platinum catalyst is Karstedt's catalyst, which is described in Karstedt's U.S. Pat. Nos. 3,715,334 and 3,814,730, incorporated herein by reference. Karstedt's catalyst is a platinum divinyl tetramethyl disiloxane complex typically containing one weight percent of platinum in a solvent such as toluene. Another preferred platinum catalyst is a reaction product of chloroplatinic acid and an organosilicon compound containing terminal aliphatic unsaturation. It is described in U.S. Pat. No. 3,419,593, incorporated herein by reference. Most preferred as the catalyst is a neutralized complex of platinous chloride and divinyl tetramethyl disiloxane, for example as described in U.S. Pat. No. 5,175,325.

One example of a suitable elastomer which may be introduced into the external phase of the present invention is a composition known in the cosmetic industry under its INCI name of Dimethicone/Vinyl Dimethicone Crosspolymer or Dimethicone Crosspolymer. Emulsions and suspension of these polysiloxane elastomers can also be used in the external phase of the present invention. Polysiloxane elastomers in the form of powders coated with different organic and inorganic materials such as mica and silica can also be used.

Carbinol Fluids: These materials are described in WO 03/101412 A2, and can be commonly described as substituted hydrocarbyl functional siloxane fluids or resins.

Water soluble or water dispersible silicone polyether compositions having an HLB of >6. These are also known as polyalkylene oxide silicone copolymers, silicone poly(oxyalkylene) copolymers, silicone glycol copolymers, or silicone surfactants. These can be linear rake or graft type materials, or ABA type where the B is the siloxane polymer block, and the A is the poly(oxyalkylene) group. The poly(oxyalkylene) group can consist of polyethylene oxide, polypropylene oxide, or mixed polyethylene oxide/polypropylene oxide groups. Other oxides, such as butylene oxide or phenylene oxide are also possible

When present the Silicone materials listed above may preferably be present in a proportion of up to about 40% of the total weight of the composition, more preferably 5 to 20%. by weight of the composition.

A composition according to the invention may also contain one or more oils as optional ingredients. The term “oil” as used herein refers to any material which is substantially insoluble in water. When the composition is to be used in a cosmetic or personal care product, the product components must also be cosmetically acceptable or otherwise meet the conditions of the end use product. Suitable oils include, but are not limited to, natural oils such as coconut oil; hydrocarbons such as mineral oil and hydrogenated polyisobutene; fatty alcohols such as octyldodecanol; esters such as C12-C15 alkyl benzoate; diesters such as propylene dipelarganate; and triesters, such as glyceryl trioctanoate. Suitable as a volatile oils component are various C8-C20 isoparaffins such as C12 isoparaffin made by The Permethyl Corporation having the tradename Permethyl® 99A, or a C12 isoparaffin (isododecane). Various C16 isoparaffins commercially available, such as isohexadecane are also suitable. The volatile solvent component can also be a mixture of volatile silicones and C8-20 isoparaffins. The one or more oils may be present in a proportion up to about 40%, more preferably from 10 to 30% by weight of the composition.

Component (i) the external phase, of the present invention may additionally comprise a proportion of optional ingredients, typically no more than 20% by weight preferably no more than 10% by weight of component (i).

The optional ingredients which may be included in component (i) may be selected from one or more of the following powders:—

Any suitable filler or combination of fillers may be utilised. The compositions may contain one or more finely divided, reinforcing fillers (e) such as high surface area fumed and precipitated silicas and to a degree calcium carbonate or additional non-reinforcing fillers such as crushed quartz, diatomaceous earths, barium sulphate, iron oxide, titanium dioxide and carbon black, talc, wollastonite. Other fillers which might be used alone or in addition to the above include aluminite, calcium sulphate (anhydrite), gypsum, calcium sulphate, magnesium carbonate, clays such as kaolin, aluminium trihydroxide, magnesium hydroxide (brucite), graphite, copper carbonate, e.g. malachite, nickel carbonate, e.g. zarachite, barium carbonate, e.g. witherite and/or strontium carbonate e.g. strontianite

Aluminium oxide, silicates from the group consisting of olivine group; garnet group; aluminosilicates; ring silicates; chain silicates; and sheet silicates. The olivine group comprises silicate minerals, such as but not limited to, forsterite and Mg₂SiO₄. The garnet group comprises ground silicate minerals, such as but not limited to, pyrope; Mg₃Al₂Si₃O₁₂; grossular; and Ca₂Al₂Si₃O₁₂. Aluninosilicates comprise ground silicate minerals, such as but not limited to, sillimanite; Al₂SiO₅; mullite; 3Al₂O₃.2SiO₂; kyanite; and Al₂SiO₅. The ring silicates group comprises silicate minerals, such as but not limited to, cordierite and Al₃(Mg,Fe)₂[Si₄AlO₁₈]. The chain silicates group comprises ground silicate minerals, such as but not limited to, wollastonite and Ca[SiO₃].

The sheet silicates group comprises silicate minerals, such as but not limited to, mica; K₂Al₁₄[Si₆Al₂O₂₀](OH)₄; pyrophyllite; Al₄[Si₈O₂₀](OH)₄; talc; Mg₆[Si₈O₂₀](OH)₄; serpentine for example, asbestos; Kaolinite; Al₄[Si₄O₁₀](OH)₈; and vermiculite.

In addition, a surface treatment of the filler(s) may be performed, for example with a fatty acid or a fatty acid ester such as a stearate, or with organosilanes, organosiloxanes, or organosilazanes hexaalkyl disilazane or short chain siloxane diols to render the filler(s) hydrophobic and therefore easier to handle and obtain a homogeneous mixture with the other sealant components The surface treatment of the fillers makes the ground silicate minerals easily wetted by the silicone polymer. These surface modified fillers do not clump, and can be homogeneously incorporated into the silicone polymer. This results in improved room temperature mechanical properties of the uncured compositions. Furthermore, the surface treated fillers give a lower conductivity than untreated or raw material.

Said fillers are present in an amount by weight from 0 to 40% preferably 0 to 25%, with respect to the weight of the final composition.

Other pulverant materials which may be used can be generally defined as dry, particulate matter having a particle size of 0.02-50 microns include glass or ceramic beads, metal soaps derived from carboxylic acids having 8-22 carbon atoms, non-expanded synthetic polymer powders, expanded powders and powders from natural organic compounds, such as cereal starches, which may or may not be cross-linked. Suitable powders include bismuth oxychloride, titanated mica, fumed silica, spherical silica beads, polymethylmethacrylate beads, micronized teflon, boron nitride, acrylate polymers, aluminium silicate, aluminium starch octenylsuccinate, bentonite, calcium silicate, cellulose, chalk, corn starch, distomaceous earth, fuller's earth, glyceryl starch, hectorite, hydrated silica, kaolin, magnesium aluminium silicate, magnesium carbonate, magnesium hydroxide, magnesium oxide, magnesium silicate, magnesium trisilicate, maltodextrin, montmorillonite, microcrystalline cellulose, rice starch, silica, talc, mica, titanium dioxide, zinc laurate, zinc myristate, zinc neodecanoate, zinc rosinate, zinc stearate, polyethylene, alumina, attapulgite, calcium carbonate, calcium silicate, dextran, kaolin, nylon, silica silylate, silk powder, serecite, soy flour, tin oxide, titanium hydroxide, trimagnesium phosphate, walnut shell powder, or mixtures thereof.

Alternatively or in addition to the above fillers colouring agents may also be utilised in the composition of the present invention, such as carbon black, chromium or iron oxides, ultramarines, manganese pyrophosphate, iron blue, and titanium dioxide, pearlescent agents. These colouring agents may be alone or in a mixture with coloured pigments and/or organic dyes. For cosmetic applications they are typically used in combination with coloured pigments. In general, when present these colouring agents can be present in an amount by weight up to 20% with respect to the weight of the final composition.

Optional Pigments which may be used in compositions in accordance with the present invention include iron oxides and titanium dioxide which, when present, are present in the composition in an amount of from 0.1 to 30 wt.-%, preferably 0.2 to 10 wt.-% and most preferably 0.4 to 2 wt.-%. Other pigments utilised might include various organic and inorganic pigments. The organic pigments are generally various aromatic types including azo, indigoid, triphenylmethane, anthraquinone, and xanthine dyes which are designated as D&C and FD&C blues, browns, greens, oranges, reds, yellows, etc. Inorganic pigments generally consist of insoluble metallic salts of certified colour additives, referred to as the Lakes or iron oxides.

The above mentioned powders may be surface treated with lecithin, amino acids, mineral oil, silicone oil, or various other agents either alone or in combination, which coat the powder surface and render the particles hydrophobic in nature.

In order to make the composition according to this invention more visually attractive, oil and water soluble colorants and glitters may be added to the composition of the present invention at a suitable level to obtain the required visual effects.

In accordance with a still further aspect of the present invention there is provided a cured product (device) resulting from the process in accordance with the present invention having a three dimensional shape, in which the minimum size of each dimension is at least 1 millimetre, preferably at least 5 millimetre, more preferably at least 1 centimetre, made from an emulsion of an aqueous phase into a 2 part silicone elastomer system using a water-in-silicone emulsifier, which is then cured, resulting into a soft rubber device of definite shape or a soft rubber sheet. Preferably such a device also comprises fragrances, essential oils, and/or water and oil soluble actives and/or silicone and organic diluents in the external silicone rubber phase for modifying the texture and feel of the device when cured.

Products prepared by the process in accordance with the present invention may be moulded into a predetermined shape by curing in a mould to form a soft solid rubber object. alternatively said product may be in the form of a flexible rubber thin sheet or a suspension of the solid rubber into water (water-in-silicone-in water suspension).

Devices in accordance with the present invention may be utilised for a wide range of uses including but not limited to fragrance delivery devices in the form of e.g. aromatherapy anti stress balls, and aromatherapy pillows; Other potential uses include for example deodorising shoe soles, solid deodorising device for houses, external prosthesis, films for wound care applications, facial mask, oral care devices requiring the delivery of actives. Devices in accordance with the present invention may also be used in the delivery of cosmetic, veterinary, pharmaceutic and therapeutic compositions for delivery of active ingredients. It can also be under the form of silicone rubber particles suspended in water (water in silicone rubber in water suspension) which could lead to potential application for encapsulation of actives.

### EXAMPLES

In order that the scope of the invention may become clearer there now follows a description of example compositions selected for description to illustrate the invention by way of example. In the description all compositions are expressed by weight % and all viscosities are measured at 25° C. unless otherwise indicated.

### Example 1

Three samples, 1.1, 1.2 and 1.3 were prepared using three alternative formulations in accordance with the present invention to prepare high water content silicone rubber devices. The same mixing program is typically used for the preparation of flexible rubber thin sheets although these were not prepared in this Example using the following steps:—
1) The external phase comprised a Dimethylhydroxy terminated polydimethylsiloxane polymer having a Viscosity of 13500 mPa·s, a Cyclopentasiloxane & PEG/PPG-18/18 Dimethicone emulsifier in each sample.
   Sample 1.1 additionally contained a further condensation curable polymer in a Triethoxysilyl ethylene terminated polydimethylsiloxane having a Viscosity of 12500 mPa·s and samples 1.2 and 1.3 contained a cyclic siloxane diluent, cyclopentasiloxane. The components of the respective external layers were intermixed in a suitable vessel.
2) An internal phase comprising water and an electrolyte (sodium chloride);
3) The internal phase was then introduced was then gradually (slowly) added into the external phase which was being mixed under turbulent agitation using a suitable mixer, typically an electrical or dental mixer;
4) After complete addition of the Internal phase mixing was continued for a further 5 minutes thereby resulting in the preparation of an emulsion;
5) The cure package was then introduced into the previously prepared emulsion and mixing continued for a further 5 minutes;
6) Any entrapped air in the composition resulting from step 5 was then removed by vacuum; and finally
7) The resulting degassed composition was poured into moulds or into a thin sheet form and was then left to reticulate (cure) for at least 24 hours. Whilst it is known curing the composition at higher temperatures would decrease the reticulation/curing time, room temperature was used. The composition in each of sample 1.1, 1.2 and 1.3 are provide for ease of reading in Table 1 below.

TABLE 1

| | Chemical composition/INCI | wt % | wt % | wt % |
|---|---|---|---|---|
| Product name | denomination | 1.1 | 1.2 | 1.3 |
| External Phase | | | | |
| Dow Corning ® | Cyclopentasiloxane & | 21.0 | 21.0 | 21.0 |
| 5225c | PEG/PPG-18/18 | | | |
| Formulation Aid | Dimethicone | | | |
| | Dimethylhydroxy terminated | 7.5 | 15.5 | 10.4 |
| | polydimethylsiloxane | | | |
| | Viscosity: 13500 mPa · s | | | |
| Cyclopentasiloxane | | | 10.0 | 6.6 |
| Trialkoxy | Triethoxysilyl ethylene | 23.5 | | |
| functionalised | terminated | | | |
| siloxane (Viscosity: | polydimethylsiloxane | | | |
| 12,500 mPa · s) | Viscosity 12500 mPa · s | | | |
| Internal Phase | | | | |
| Sodium Chloride | | 1.35 | 1.35 | 1.35 |
| Water | | 46.25 | 48.65 | 57.15 |
| Cure Package | | | | |
| Tetraethoxysilane | Tetroorthosilicate | | 3.0 | 3.0 |
| (TEOS) | | | | |
| Fomrez ® UL- | Dimethyl tin dineodecanoate | 0.4 | 0.5 | 0.5 |
| 28 (from | | | | |
| Nordmann | | | | |
| Rassmann | | | | |
| GmbH, Hamburg, | | | | |
| Germany | | | | |

The inventors found that the resulting cured products had different textures dependent on the composition used. The cured product of Sample 1.1 was considered to have soft to hard rubber device leaving a refreshing wet feel when handled The cured product of Sample 1.2 was considered to have hard solid texture with very wet feel, low resistance to tear, white homogeneous colour, non homogeneous shape due to a high viscosity before the reticulation step.

The cured product of Sample 1.3 was considered to have a hard texture with very wet feel, good resistance to tear, White homogeneous colour, non homogeneous shape due to a high viscosity before the reticulation step.

### Example 2

In this example there is provided sample 2.1 which when cured provided a Water containing Silicone solid device with high level of essential oil in accordance with the present invention. The composition was prepared using the same process as described in Example 1 although the internal phase additionally contained lavender essential oil as opposed to merely water and electrolyte. The composition of sample 2.1 is shown in Table 2 below

TABLE 2

| | Chemical composition/INCI | Wt % |
|---|---|---|
| Product name | denomination | 2.1 |
| External Phase | | |
| Dow Corning ® 5225c | Cyclopentasiloxane & PEG/PPG- | 21.0 |
| Formulation Aid | 18/18 Dimethicone | |
| | Dimethylhydroxy terminated | 31 |
| | polydimethylsiloxane | |
| | Viscosity: 13500 mPa · s | |
| Internal Phase | | |
| Sodium Chloride | | 1.35 |
| Lavender essential oil | | 20 |
| Water | | 23.15 |
| Cure Package | | |
| TEOS | Tetraethylorthosilicate | 3.0 |
| Fomrez ® UL-28 | Dimethyl tin dineodecanoate | 0.5% |

The resulting cured product had a hard and nice texture without any tackiness and the aroma of the lavender oil could be detected nasally for a period of over 2 months.

### Example 3

In this example three alternate Water containing silicone rubber products were prepared. The compositions contained relatively high levels of Perfume of different polarity which is introduced into the composition as part of the internal phase. Samples were prepared using the process described in Example 1 above and the composition of each sample 3.1 to 3.3 is depicted in Table 3 below:—

TABLE 3

| | Chemical composition/ | wt % | wt % | wt % |
|---|---|---|---|---|
| Product name | INCI denomination | 3.1 | 3.2 | 3.3 |
| External Phase | | | | |
| Dow Corning ® 5225c | Cyclopentasiloxane & | 21.0 | 21.0 | 21.0 |
| | PEG/PPG-18/18 | | | |
| | Dimethicone | | | |
| | dimethylhydroxyl- | 31.0 | 31.0 | 31.0 |
| | terminated | | | |
| | diorganopolysiloxane | | | |
| | Viscosity: 13500 mPa · s | | | |
| Internal Phase | | | | |
| Sodium Chloride | | 1.35 | 1.35 | 1.35 |
| Medium to high polarity | | 20.0 | | |
| perfume (Zen*). | | | | |
| Medium polarity perfume | | | 20.0 | |
| (Musk*) | | | | |
| High polarity perfume | | | | 20.0 |
| (Gourmande*) | | | | |
| Water | | 23.15 | 23.15 | 23.15 |
| Cure Package | | | | |
| TEOS | Tetraethylorthosilicate | 3.0 | 3.0 | 3.0 |
| Fomrez ® UL-28 | Dimethyl tin | 0.5 | 0.5 | 0.5 |
| | dineodecanoate | | | |

All the Perfumes used were provided courtesy of Symrise

Sample 3.1, when cured was found to have a Soft to hard texture, low resistance to tear, and a slightly greasy feel, but had homogeneous colour and shape; Sample 3.2 when cured was found to have a: Hard texture, which was quite resistant to tear, Slightly greasy feel, off white homogeneous colour, non homogeneous shape due to a high viscosity before the reticulation step; and Sample 3.3 when cured was found to have a hard texture, which was quite resistant to tear, wet and greasy feel, yellowish homogeneous colour, non homogeneous shape due to a high viscosity before the reticulation step.

In each case it was found that perfume could be nasally detected for periods of greater than 1 month.

### Example 4

This example was undertaken to show that a number of commonly used perfume solvents could be retained by the rubber product of the present invention, when cured. Samples 4.1, 4.2 and 4.3 were each prepared in accordance with the process followed in Example 1 above. The compositions used for each sample are depicted in Table 4 below.

TABLE 4

| | Chemical composition/ | wt % | wt % | wt % |
|---|---|---|---|---|
| Product name | INCI denomination | 4.1 | 4.2 | 4.3 |
| External Phase | | | | |
| Dow Corning ® 5225c | Cyclopentasiloxane & | 21.0 | 21.0 | 21.0 |
| Formulation Aid | PEG/PPG-18/18 | | | |
| | Dimethicone | | | |
| | Dimethylhydroxy | 31.0 | 31.0 | 31.0 |
| | terminated | | | |
| | polydimethylsiloxane | | | |
| | Viscosity: 13500 mPa · s | | | |
| Internal Phase | | | | |
| Sodium Chloride | | 1.35 | 1.35 | 1.35 |
| Triethyl citrate | | 20.0 | | |
| Dipropylene Glycol | | | 20.0 | |
| Isopropyl Myristate. | | | | 20.0 |
| Water | | 23.15 | 23.15 | 23.15 |
| Cure Package | | | | |
| TEOS | Tetraethylorthosilicate | 3.0 | 3.0 | 3.0 |
| Fomrez ® UL-28 | Dimethyl tin | 0.5 | 0.5 | 0.5 |
| | dineodecanoate | | | |

Sample 4.1 when cured was found to have a Soft to hard white solid texture with some exudation coming out, with homogeneous shape. Sample 4.2 when cured was found to have a Translucent soft to hard solid texture with more important exudation, non homogeneous shape due to a very high viscosity before reticulation; and Sample 4.3 when cured was found to have a Hard, white homogeneous solid texture with no exudation, homogeneous shape.

### Example 5

This example was undertaken to show that products in accordance with the present invention can be obtained when incorporating a variety of complex perfume in the respective compositions. The composition was prepared using the same process as described in Example 1. The composition of samples 5.1 to 5.4 are shown below in Table 5

TABLE 5

| | Chemical composition/ | wt % | wt % | wt % | wt % |
|---|---|---|---|---|---|
| Product name | INCI denomination | 5.1 | 5.2 | 5.3 | 5.4 |
| External Phase | | | | | |
| Dow Corning ® | Cyclopentasiloxane & | 21.0 | 21.0 | 21.0 | 21.0 |
| 5225c | PEG/PPG-18/18 | | | | |
| Formulation Aid | | | | | |
| | Dimethicone | | | | |
| | Dimethylhydroxy | 31.0 | 31.0 | 31.0 | 31.0 |
| | terminated | | | | |
| | polydimethylsiloxane | | | | |
| | Viscosity: 13500 mPa · s | | | | |
| Internal Phase | | | | | |
| Sodium Chloride | | 1.35 | 1.35 | 1.35 | 1.35 |
| Pinacolada* | | 20.0 | | | |
| Legoria senso* | | | 20.0 | | |
| Summer fruit | | | | 20.0 | |
| cocktail. | | | | | |
| Vanille* | | | | | 20.0 |
| Water | | 23.15 | 23.15 | 23.15 | 23.15 |
| Cure Package | | | | | |
| TEOS | Tetraethylorthosilicate | 3.0 | 3.0 | 3.0 | 3.0 |
| Fomrez ® UL-28 | Dimethyl tin | 0.5 | 0.5 | 0.5 | 0.5 |
| | dineodecanoate | | | | |

*Perfumes, courtesy of Symrise

Sample 5.1 when cured was found to have a: hard yellowish, resistant solid texture with perfume exudation and greasy feel, non homogeneous shape due to a high viscosity before reticulation. Sample 5.2 when cured was found to have a Medium hard, yellowish quite resistant solid texture with some perfume exudation and greasy feel, non homogeneous shape due to a very high viscosity before reticulation. Sample 5.3 when cured was found to have a hard white, translucent white, brittle solid texture with some exudation with greasy and tacky feel, non homogeneous shape due to a very high viscosity before reticulation. Sample 5.4 when cured was found to be the same as sample 5.3.

In each case it was found that perfume could be nasally detected for periods of greater than 1 month.

### Example 6

This example was undertaken to show that products in accordance with the present invention can be obtained when incorporating a blend of perfume and essential oils in sample 6.1. The sample was prepared using the same process as described in Example 1. The composition of sample 6.1 is shown below in Table 6

TABLE 6

| | Chemical composition/INCI | wt % |
|---|---|---|
| Product name | denomination | 6.1 |
| External Phase | | |
| Dow Corning ® | Cyclopentasiloxane & | 21.0 |
| 5225c | PEG/PPG-18/18 | |
| Formulation Aid | Dimethicone | |
| | Dimethylhydroxy terminated | 31.0 |
| | polydimethylsiloxane | |
| | Viscosity: 13500 mPa · s. | |
| Internal Phase | | |
| Sodium Chloride | | 1.35 |
| Vanille* | | 10.0 |
| Lavender essential oil | | 10.0 |
| Water | | 23.15 |
| Cure Package | | |
| TEOS | Tetraethylorthosilicate | 3.0 |
| Fomrez ® UL-28 | Dimethyl tin dineodecanoate | 0.5 |

*Perfume, courtesy of Symrise

Sample 6.1 when cured was found to have a Hard, yellowish, low resistance solid texture with some perfume exudation and slippery feel, wet feel, homogeneous shape. Release of the perfume lasted for a period >1 month.

### Example 7

This example, like example 2, shows the use of lavender essential oil in the internal phase of the composition but depicts compositions 7.1 to 7.4 with different levels of cross-linker in the curing package to determine the variation in texture of the final product caused by the effect of varying levels of cross-linker. The samples was prepared using the same process as described in Example 1.

| | Chemical composition/ | wt % | wt % | wt % | wt % |
|---|---|---|---|---|---|
| Product name | INCI denomination | 7.1 | 7.2 | 7.3 | 7.4 |
| External Phase | | | | | |
| Dow Corning ® | Cyclopentasiloxane & | 21.0 | 21.0 | 21.0 | 21.0 |
| 5225c | PEG/PPG-18/18 | | | | |
| Formulation Aid | | | | | |
| | Dimethicone | | | | |
| | Dimethylhydroxy | 31.0 | 31.0 | 31.0 | 31.0 |
| | terminated | | | | |
| | polydimethylsiloxane | | | | |
| | Viscosity: 13500 mPa · s | | | | |
| Internal Phase | | | | | |
| Sodium Chloride | | 1.35 | 1.35 | 1.35 | 1.35 |
| Lavender | | 20.0 | 20.0 | 20.0 | 20.0 |
| essential oil* | | | | | |
| Water | | 25.15 | 24.15 | 23.15 | 22.15 |
| Cure Package | | | | | |
| TEOS | Tetraethylorthosilicate | 1.0 | 2.0 | 3.0 | 4.0 |
| Fomrez ® UL-28 | Dimethyl tin | 0.5 | 0.5 | 0.5 | 0.5 |
| | dineodecanoate | | | | |

Sample 7.1 when cured had a Very soft white solid texture with a slightly tacky feel. No exudation and homogeneous shape; Sample 7.2 when cured had a Soft white solid texture with no tacky feel. No exudation and homogeneous shape. Sample 7.3 when cured had a hard white solid texture, quite resistant with no exudation and no tacky feel. Homogeneous shape. Sample 7.4 when cured had a Very hard white texture, quite resistant with no exudation and no tacky feel. Homogeneous shape. Release of the perfume lasted for a period >than 3 months.

### Example 8

This example was undertaken to consider the variation caused by the change in emulsifier in the external phase. The samples were prepared using the same process as described in Example 1. The compositions of samples 8.1 to 8.5 are depicted in Table 8 below

TABLE 8

| | Chemical composition/ | wt % | wt % | wt % | wt % | wt % |
|---|---|---|---|---|---|---|
| Product name | INCI denomination | 8.1 | 8.2 | 8.3 | 8.4 | 8.5 |
| External Phase | | | | | | |
| Dow Corning ® 5200 | Lauryl PEG/PPG- | 4.0 | | | | |
| | 18/18 Methicone | | | | | |
| Dow Corning ® | Cyclopentasiloxane & | | 4.0 | | | |
| By11-030 | PEG/PPG 19/19 | | | | | |
| | Dimethicone | | | | | |
| Dow Corning ® 9011 | Cyclopentasiloxane & | | | 21.0 | | |
| | PEG-12 Dimethicone cross polymer. | | | | | |
| Abil EM 90 | Cetyl PEG/PPG-10/1 | | | | 4.0 | |
| (Degussa) | Dimethicone | | | | | |
| Abil EM 97 | Bis-PEG/PPG-14/14 | | | | | 3.0 |
| (Degussa) | Dimethicone & | | | | | |
| | cyclopentasiloxane | | | | | |
| | Dimethylhydroxy | 31.0 | 31.0 | 31.0 | 31.0 | 31.0 |
| | terminated | | | | | |
| | polydimethylsiloxane | | | | | |
| | Viscosity: 13500 mPa · s | | | | | |
| Cyclopentasiloxane | | 37.0 | 37.0 | 20.0 | 37.0 | 38.0 |
| Internal Phase | | | | | | |
| Sodium Chloride | | 1.35 | 1.35 | 1.35 | 1.35 | 1.35 |
| Water | | 23.15 | 23.15 | 23.15 | 23.15 | 23.15 |
| Cure Package | | | | | | |
| TEOS | Tetraethylorthosilicate | 3.0 | 3.0 | 3.0 | 3.0 | 3.0 |
| Fomrez ® UL-28 | Dimethyl tin | 0.5 | 0.5 | 0.5 | 0.5 | 0.5 |
| | dineodecanoate | | | | | |

Sample 8.1 when cured had a Soft to hard solid texture with wet refreshing feel, low resistance, homogeneous shape and colour. Sample 8.2 when cured had a Soft to hard solid texture with wet and refreshing feel, quite resistant, very homogeneous colour and shape. Sample 8.3 when cured had a Hard solid texture with slightly wet and refreshing feel, quite resistant, very homogeneous colour but less shiny than example 2. Sample 8.4 when cured had a Soft to hard solid texture with wet and refreshing feel, not so resistant, very homogeneous colour but not shape. Sample 8.5 when cured had a Soft to hard solid texture with wet and refreshing feel, quite resistant, very homogeneous colour and shape These 5 compositions demonstrate that different silicone emulsifiers can be used to make these water containing silicone rubber in accordance with the present invention.

### Example 9

This example was undertaken to consider the effect of varying the polymer in the external phase of the composition. The samples were prepared using the same process as described in Example 1. The compositions of samples 9.1 to 9.4 are depicted in Table 9 below.

TABLE 9

| | Chemical composition/ | wt % | wt % | wt % | wt % |
|---|---|---|---|---|---|
| Product name | INCI denomination | 9.1 | 9.2 | 9.3 | 9.4 |
| External Phase | | | | | |
| Dow Corning ® | Cyclopentasiloxane & | 21 | 21 | 21 | 21 |
| 5225c | PEG/PPG 18/18 | | | | |
| Formulation Aid | | | | | |
| | Dimethicone | | | | |
| | Dimethylhydroxy | 31.0 | | | |
| | terminated | | | | |
| | polydimethylsiloxane | | | | |
| | Viscosity: | | | | |
| | 13500 mPa · s | | | | |
| | Dimethylhydroxy | | 31.0 | | |
| | terminated | | | | |
| | polydimethylsiloxane | | | | |
| | Viscosity: | | | | |
| | 4000 mPa · s | | | | |
| | Dimethylhydroxy | | | 31.0 | 29.0 |
| | terminated | | | | |
| | polydimethylsiloxane | | | | |
| | Viscosity: | | | | |
| | 2000 mPa · s | | | | |
| Dow Corning ® | Dimethiconol | | | | 2.0 |
| 1254 | | | | | |
| fluid (PA Fluid) | | | | | |
| Cyclopentasiloxane | | 20.0 | 20.0 | 20.0 | 20.0 |
| Internal Phase | | | | | |
| Sodium Chloride | | 1.35 | 1.35 | 1.35 | 1.35 |
| Water | | 23.15 | 23.15 | 23.15 | 23.15 |
| Cure Package | | | | | |
| TEOS | Tetraethylorthosilicate | 3.0 | 3.0 | 3.0 | 3.0 |
| Fomrez ® UL-28 | Dimethyl tin | 0.5 | 0.5 | 0.5 | 0.5 |
| | dineodecanoate | | | | |

Sample 9.1 when cured had a hard solid texture with exudation, very white & quite shiny with a good resistance. Homogeneous colour and shape. Sample 9.2 when cured had a hard solid texture with wet and refreshing feel, low resistance, very homogeneous colour and shape. Sample 9.3 when cured had a: Quite hard solid texture with wet and refreshing feel, low resistance, quite fragile, very homogeneous colour and shape. Sample 9.1 when cured was found to be substantially the same texture etc. as noted for 9.3. These 4 compositions demonstrate that the type of reactive OH end blocked polymer has an impact on the hardness and resistance of the water containing silicone rubber.

### Example 10

This example was undertaken to consider the effect of varying the filler in the external phase of the composition. The samples were prepared using the same process as described in Example 1. The compositions of samples 10.1 to 10.5 are depicted in Table 10 below.

TABLE 10

| | Chemical composition/ | wt % | wt % | wt % | wt % | wt % |
|---|---|---|---|---|---|---|
| Product name | INCI denomination | 10.1 | 10.2 | 10.3 | 10.4 | 10.5 |
| External Phase | | | | | | |
| Dow Corning ® | Cyclopentasiloxane & | 21.0 | 21.0 | 21.0 | 21.0 | 21.0 |
| 5225c | PEG/PPG 18/18 | | | | | |
| Formulation Aid | Dimethicone | | | | | |
| | Dimethylhydroxy | 26.0 | 26.0 | 21.0 | 11.0 | 6.0 |
| | terminated | | | | | |
| | polydimethylsiloxane | | | | | |
| | Viscosity: 13500 mPa · s | | | | | |
| SOCAL ® 31 | Precipitated calcium | 5.0 | | 10.0 | 20.0 | 25.0 |
| (Solvay): treated calcium | Carbonate treated | | | | | |
| Carbonate | with stearic acid | | | | | |
| CAB-O-SIL ® | fumed silica treated | | 5.0 | | | |
| TS-530 | with | | | | | |
| | hexamethyldisilazane | | | | | |
| Cyclopenta-siloxane | | 20.0 | 20.0 | 20.0 | 20.0 | 20.0 |
| Internal Phase | | | | | | |
| Sodium Chloride | | 1.35 | 1.35 | 1.35 | 1.35 | 1.35 |
| Water | | 23.15 | 23.15 | 23.15 | 23.15 | 23.15 |
| Cure Package | | | | | | |
| TEOS | Tetraethylorthosilicate | 3.0 | 3.0 | 3.0 | 3.0 | 3.0 |
| Fomrez ® UL-28 | Dimethyl tin | 0.5 | 0.5 | 0.5 | 0.5 | 0.5 |
| | dineodecanoate | | | | | |

Sample 10.1 when cured had a Soft to hard solid texture with dry feel, not very resistant, homogeneous colour and shape. Sample 10.2 when cured had a hard solid texture with very dry feel, homogeneous colour and shape, good resistance. Sample 10.3 when cured had a Soft to hard solid texture with dry feel, not very resistant, homogeneous colour and shape. Sample 10.4 when cured had a Soft, elastic solid texture with dry feel, quite resistant, homogeneous and very white colour and shape Sample 10.5 when cured had a Soft, elastic solid texture with dry feel, quite resistant, homogeneous and very white colour and shape. These 5 compositions demonstrate that the impact of the filler on the hardness and the resistance of the water containing silicone rubber.

### Example 11

This example was undertaken to consider the effect of differing non-reactive silicone based diluents in the external phase of the composition. The samples were prepared using the same process as described in Example 1. The compositions of samples 11.1 to 11.6 are depicted in Table 11 below.

TABLE 11

| | Chemical | | | | | | |
|---|---|---|---|---|---|---|---|
| | composition/INCI | wt % | wt % | wt % | wt % | wt % | wt % |
| Product name | denomination | 11.1 | 11.2 | 11.3 | 11.4 | 11.5 | 11.6 |
| External phase | | | | | | | |
| Dow Corning ® | Cyclopentasiloxane | 21.0 | 21.0 | 21.0 | 21.0 | 21.0 | 21.0 |
| 5225c | & PEG/PPG 18/18 | | | | | | |
| Formulation | Dimethicone | | | | | | |
| Aid | | | | | | | |
| | Dimethylhydroxy | 31.0 | 31.0 | 31.0 | 31.0 | 31.0 | 31.0 |
| | terminated | | | | | | |
| | polydimethyl- | | | | | | |
| | siloxane, Viscosity: | | | | | | |
| | 13500 mPa · s | | | | | | |
| Dow Corning ® | Phenyl | 20.0 | | | | | |
| 556 | Trimethicone | | | | | | |
| Dow Corning ® | Bis-hydroxyethoxy- | | 20.0 | | | | |
| 5562 | propyl Dimethicone | | | | | | |
| Dow Corning ® | Dimethicone & | | | 20.0 | | | |
| 1184 | Trisiloxane | | | | | | |
| Dow Corning ® | Cyclopentasiloxane | | | | 20.0 | | |
| 670 | & polypropyl- | | | | | | |
| | silesquioxane | | | | | | |
| Dow Corning ® | Dimethicone | | | | | 20.0 | |
| 200 fluid 5 mPa · s | | | | | | | |
| Dow Corning ® 9045 | cyclopentasiloxane | | | | | | 20.0 |
| | & Dimethicone | | | | | | |
| | Crosspolymer | | | | | | |
| Internal Phase | | | | | | | |
| Sodium | | 1.35 | 1.35 | 1.35 | 1.35 | 1.35 | 1.35 |
| Chloride | | | | | | | |
| Water | | 23.15 | 23.15 | 23.15 | 23.15 | 23.15 | 23.15 |
| Cure Package | | | | | | | |
| TEOS | Tetraethylortho- | 3.0 | 3.0 | 3.0 | 3.0 | 3.0 | 3.0 |
| | silicate | | | | | | |
| Fomrez ® UL- | Dimethyl tin | 0.5 | 0.5 | 0.5 | 0.5 | 0.5 | 0.5 |
| 28 | dineodecanoate | | | | | | |

Sample 11.1 when cured had a: Hard solid texture with dry feel, resistant, homogeneous colour and shape. Sample 11.2 when cured had a hard solid texture with greasy feel and exudation. Homogeneous colour and shape, good resistance. Sample 11.3 when cured had a: Same as sample 11.1 Sample 11.4 when cured had a: very hard solid texture with dry feel and good resistance. Homogeneous colour and shape. Sample 11.5 when cured had a: Soft to hard solid texture with a very dry feel, quite low resistance to tear. Homogeneous colour and shape Sample 11.6 when cured had a: Soft to hard solid texture with a very dry feel, quite low resistance to tear. Homogeneous colour and shape all these composition are showing that the addition of different types of silicone materials has an impact on the hardness and the resistance of the water containing silicone rubber.

### Example 12

This example was undertaken to consider the effect of including cosmetic oils in the external phase of the composition. The samples were prepared using the same process as described in Example 1. The compositions of samples 12.1 to 12.7 are depicted in Table 12 below.

TABLE 12

| | Chemical | | | | | | | |
|---|---|---|---|---|---|---|---|---|
| Product | composition/INCI | wt % | wt % | wt % | wt % | wt % | wt % | Wt % |
| name | denomination | 12.1 | 12.2 | 12.3 | 12.4 | 12.5 | 12.6 | 12.7 |
| External Phase | | | | | | | | |
| Dow | cyclopentasiloxane | 21.0 | 21.0 | 21.0 | 21.0 | 21.0 | 21.0 | 21.0 |
| Corning ® | & PEG/PPG 18/18 | | | | | | | |
| 5225c | Dimethicone | | | | | | | |
| Formulation | | | | | | | | |
| Aid | | | | | | | | |
| | Dimethylhydroxy | 31.0 | 31.0 | 31.0 | 31.0 | 31.0 | 31.0 | 31.0 |
| | terminated | | | | | | | |
| | polydimethyl- | | | | | | | |
| | siloxane | | | | | | | |
| | Viscosity: 13500 mPa · s | | | | | | | |
| Isododecane | | 20.0 | | | | | | |
| Isohexadecane | | | 20.0 | | | | | |
| Polyisobutene | | | | 20.0 | | | | |
| Isopropyl | | | | | 20.0 | | | |
| Palmitate | | | | | | | | |
| C12-15 | | | | | | 20.0 | | |
| Alkylbenzoate. | | | | | | | | |
| Capryl | | | | | | | 20.0 | |
| Caprylic | | | | | | | | |
| triglycerides | | | | | | | | |
| Mineral oil | | | | | | | | 20.0 |
| Internal Phase | | | | | | | | |
| Sodium | | 1.35 | 1.35 | 1.35 | 1.35 | 1.35 | 1.35 | 1.35 |
| Chloride | | | | | | | | |
| Water | | 23.15 | 23.15 | 23.15 | 23.15 | 23.15 | 23.15 | 23.15 |
| Cure Package | | | | | | | | |
| TEOS | Tetraethylortho- | 3.0 | 3.0 | 3.0 | 3.0 | 3.0 | 3.0 | 3.0 |
| | silicate | | | | | | | |
| Fomrez ® UL- | Dimethyl tin | 0.5 | 0.5 | 0.5 | 0.5 | 0.5 | 0.5 | 0.5 |
| 28 | dineodecanoate | | | | | | | |

Sample 12.1 when cured had a hard solid texture with dry feel, low resistance, homogeneous colour and shape. Sample 12.2 when cured had a: Hard solid texture with dry feel, resistant. Homogeneous colour and shape. Sample 12.3 when cured had a: Hard solid texture quite brittle with dry feel. Very white homogeneous colour and homogeneous shape. Sample 12.4 when cured had a: very hard solid texture with dry feel and good resistance. Homogeneous colour and shape. Sample 12.5 when cured had a: Soft to hard solid texture quite brittle with dry, soft feel. Homogeneous colour and shape Sample 12.6 when cured had a: Quite hard solid texture with a greasy feel and a good resistance to tear. Homogeneous colour and shape. Sample 12.7 when cured had a: Hard solid texture with a wet feel and exudation. Low resistance. Homogeneous colour and shape. All these composition are showing that the addition of organic oils an impact on the hardness and the resistance of the water containing silicone rubber.

### Example 13

This example was undertaken to consider the effect of varying the type of emulsifier in the external phase of the composition. The samples were prepared using the same process as described in Example 1. The compositions of samples 13.1 to 13.4 are depicted in Table 13 below.

TABLE 13

| | Chemical composition/INCI | wt % | wt % | wt % | wt % |
|---|---|---|---|---|---|
| Product name | denomination | 13.1 | 13.2 | 13.3 | 13.4 |
| External Phase | | | | | |
| Dow Corning ® 5225c | Cyclopentasiloxane & PEG/PPG- | 6.0 | | | |
| Formulation Aid | 18/18 Dimethicone | | | | |
| Dow Corning ® 9011 | Cyclopentasiloxane (and) PEG- | | 2.0 | | |
| Silicone Elastomer | 12 Dimethicone Crosspolymer | | | | |
| blend | | | | | |
| Dow Corning ® | Cyclopentasiloxane (and) | | | 2.0 | |
| BY11-030 | PEG/PPG-19/19 Dimethicone | | | | |
| Emulsifier/gelling agent | | | | | |
| Dow Corning ® FZ-2233 | PEG/PPG 10/7 Dimethicone | | | | 0.5 |
| | Copolymer. | | | | |
| | Dimethylhydroxy terminated | 31.0 | 31.0 | 31.0 | 31.0 |
| | polydimethylsiloxane | | | | |
| | Viscosity: 13500 mPa · s | | | | |
| Cyclopentasiloxane | | 35.0 | 35.0 | 39.0 | 40.5 |
| Interior phase | | | | | |
| Sodium Chloride | | 1.35 | 1.35 | 1.35 | 1.35 |
| Water | | 23.15 | 27.15 | 23.15 | 23.15 |
| Cure Package | | | | | |
| TEOS | Tetraethylorthosilicate | 3.0 | 3.0 | 3.0 | 3.0 |
| Fomrez ® UL-28 | Dimethyl tin dineodecanoate | 0.5 | 0.5 | 0.5 | 0.5 |

Sample 13.1 when cured had a Soft, nice texture, white homogeneous appearance, dry feel Sample 13.2 when cured had a Soft, nice texture, white homogeneous appearance, slightly wet feel Sample 13.3 when cured had a Soft, nice texture, white homogeneous appearance, dry feel Sample 13.4 when cured had a Soft, nice texture, supple, white homogeneous appearance, dry feel All these composition are showing that several type and level of emulsifiers can be used to make good quality water containing silicone rubber products in accordance with the present invention.

### Example 14

This example was undertaken to assess whether water can be replaced by propylene glycol in the internal phase of the present invention. The samples were prepared using the same process as described in Example 1 with the exception that the active ingredient was introduced into the composition as part of the external phase. The compositions of comparative samples 14.1 (c) to 14.3 (c) and sample 14.4 are depicted in Table 14 below.

| | | wt % | wt % | wt % | wt % |
|---|---|---|---|---|---|
| | Chemical composition/INCI | 14.1 | 14.2 | 14.3 | 14.4 |
| Product name | denomination | (c) | (c) | (C) | |
| External Phase | | | | | |
| Dow Corning ® FZ-2233 | PEG/PPG 10/7 Dimethicone | 0.5 | 0.5 | 0.5 | 0.5 |
| | Copolymer. | | | | |
| | Dimethylhydroxy terminated | 31.0 | 31.0 | 31.0 | 31.0 |
| | polydimethylsiloxane | | | | |
| | Viscosity: 13500 mPa · s | | | | |
| Cyclopentasiloxane | | 40.5 | 40.0 | 35.5 | 35.5 |
| Pigments | | | 0.5 | | |
| Perfume | | | | 5.0 | 5.0 |
| Internal Phase | | | | | |
| Sodium Chloride | | 1.35 | 1.35 | 1.35 | 1.35 |
| Propylene Glycol | | 23.15 | 23.15 | 23.15 | |
| water | | | | | 23.15 |
| Cure Package | | | | | |
| TEOS | Tetraethylorthosilicate | 3.0 | 3.0 | 3.0 | 3.0 |
| Fomrez ® UL-28 | Dimethyl tin dineodecanoate | 0.5 | 0.5 | 0.5 | 0.5 |

Comparative Sample 14.1 when cured was noted to have Very soft, tacky texture with a wet feel and unlike all the previous examples exudation of the propylene glycol was seen within a period of 5 days from cure. Similarly Comparative Sample 14.2 when cured had an immediate (Direct) exudation of propylene glycol from the cured solid

Comparative Sample 14.3 when cured had the same problem as Comparative sample 14.2 above in that immediate (Direct) exudation of propylene glycol from the solid. These Comparative Samples demonstrate that although it is possible to replace the water in the internal phase of the composition with propylene glycol, there was a clear compatibility problem at the levels of addition used with the quality of the silicone rubber being less acceptable and less flexible towards the addition of pigments and perfume. However, example 14.4 in which the propylene glycol was replaced by water gave excellent results.

### Example 15

This example was undertaken to consider the compatibility of detergent in the internal phase of the composition. The samples were prepared using the same process as described in Example 1. The compositions of samples 15.1 and 15.2 are depicted in Table 15 below.

| | Chemical composition/INCI | wt % | wt % |
|---|---|---|---|
| Product name | denomination | 15.1 | 15.2 |
| External Phase | | | |
| Dow Corning ® FZ-2233 | PEG/PPG 10/7 Dimethicone | 0.5 | |
| | Copolymer. | | |
| Dow Corning ® 5225c | Cyclopentasiloxane (and) | | 21.0 |
| Formulation Aid | PEG/PPG-18/18 Dimethicone | | |
| formulation aid | | | |
| | Dimethylhydroxy terminated | 31.0 | 31.0 |
| | polydimethylsiloxane | | |
| | Viscosity: 13500 mPa · s | | |
| Cyclopentasiloxane | | 40.5 | 20.0 |
| Internal Phase | | | |
| Sodium Chloride | | 1.35 | 1.35 |
| Oramix ® NS 10 (30% in | Decyl Glucoside (Seppic) | 23.15 | 23.15 |
| water) | | | |
| Cure Package | | | |
| TEOS | Tetraethylorthosilicate | 3.0 | 3.0 |
| Fomrez ® UL-28 | Dimethyl tin dineodecanoate | 0.5 | 0.5 |

Sample 15.1 when cured had a soft texture, light wet feel. Sample 15.2 when cured had a soft texture, dry feel. These compositions demonstrate that it is possible to add a detergent to the internal phase of the silicone rubber which could be used as a cleansing device.

### Example 16

This example was undertaken to consider the variation of pH of water in the internal phase of the composition in accordance with the present invention. The compositions of samples 16.1 and 16.2 are depicted in Table 16 below. These samples were prepared using the process described in example with the exception of the following:
i) after the introduction of the internal phase to form an emulsion a pH modifier was introduced into the emulsion; and
ii) after addition of the cure package was introduced into the pre-forme pH adjusted emulsion, the samples were degasified as in Example 1 and the resulting degassed emulsions were poured into suitable moulds and cured at room temperature for 1 hour.

TABLE 16

| | Chemical composition/INCI | Wt % | Wt % |
|---|---|---|---|
| Product name | denomination | 16.1 | 16.2 |
| External Phase | | | |
| Dow Corning ® | Cyclopentasiloxane (and) | 21.0 | 21.0 |
| 5225c | PEG/PPG-18/19 Dimethicone | | |
| Formulation Aid | | | |
| formulation aid | | | |
| | Dimethylhydroxy terminated | 31.0 | 31.0 |
| | polydimethylsiloxane | | |
| | Viscosity: 13500 mPa · s | | |
| Cyclopentasiloxane | | 20.0 | 20.0 |
| Internal Phase | | | |
| Sodium Chloride | | 1.35 | 1.35 |
| Water | | 23.15 | 23.15 |
| pH modifier | | | |
| NaOH (10% in water) | | q.s. pH | |
| | | 9.0 | |
| Citric Acid (10% in | | | q.s. pH |
| water) | | | 3.0 |
| Cure Package | | | |
| TEOS | Tetraethylorthosilicate | 3.0 | 3.0 |
| Fomrez ® UL-28 | Dimethyl tin dineodecanoate | 0.5 | 0.5 |

Sample 16.1 when cured had a soft texture, dry feel, no tacky, more fragile. Sample 16.2 when cured had a soft texture, dry feel, no tacky. These compositions demonstrate that it is possible to make water containing silicone rubber at both pH acid and pH basic.

### Example 17

This example was undertaken to exemplify a method for introducing pre-cured silicone rubber particles into emulsions in accordance with the present invention. The compositions of samples 17.1 and 17.2 are depicted in Table 17 below. To achieve such a composition a slightly different process was utilised

Procedure:

### For the “Primary” Emulsion:

Mix External phase ingredients together.
Mix internal phase ingredients together.
Introduce the internal phase into the external phase very slowly while turbulently mixing at approximately 1300 RPM.

For the “Secondary” emulsion:

⁽¹⁾Mix the components of external phase (2) together at 400 RPM

Add this mixture drop by drop, very slowly to internal phase (2) and then add the cure package and cure for one hour.

TABLE 17

| | Chemical composition/INCI | wt % | wt % |
|---|---|---|---|
| Product name | denomination | 17.1 | 17.2 |

A

| | | | |
|---|---|---|---|
| Primary emulsion | | | |
| External Phase | | | |
| Dow Corning ® 5225c | Cyclopentasiloxane (and) | 21.0 | 21.0 |
| Formulation Aid | PEG/PPG-18/19 Dimethicone | | |
| | Dimethylhydroxy terminated | 31.0 | 31.0 |
| | polydimethylsiloxane | | |
| | Viscosity: 13500 mPa.s | | |
| Cyclopentasiloxane | | 20.0 | 20.0 |
| Internal Phase | | | |
| Sodium Chloride | | 1.35 | 1.35 |
| Water | | 23.15 | 23.15 |

B

| | | | |
|---|---|---|---|
| Secondary emulsion | | | |
| Internal phase (2) | | | |
| Primary emulsion | | 67.0 | 67.0 |
| Rhodasurf ® L-7 90 | Sodium C12-15 Pareth Sulfate | 3.0 | 3.0 |
| | (Rhodia) | | |
| Fomrez ® UL-28 | Dimethyl tin dineodecanoate | | 0.5 |
| External Phase (2) | | | |
| Water | | 30 | 30 |
| Cure Package | | | |
| TEOS | Tetraethylorthosilicate | 3.0 | 3.0 |
| Fomrez ® UL-28 | Dimethyl tin dineodecanoate | 0.5 | |

Sample 17.1 and sample 17.2 appeared solid after curing but investigations showed them to be very fragile breaking as soon as manipulated demonstrating that there are 2 water phases, one internal and one external.

1. A process for the production of a water-containing silicone rubber device comprising the steps of mixing
I. an external phase comprising an organopolysiloxane polymer having at least two reactive groups per molecule, a water in oil emulsifier and where required one or more active ingredients and/or optional additives; with
II. an internal phase comprising water and optionally one or more active ingredients to form an emulsion; and subsequently introducing
III. a cure package comprising a suitable catalyst and optionally a cross-linker, which catalyst will be determined by the cure reaction of (I) or the cure reaction between (I) and the cross-linker where present, and where the cross-linker contains more than two reactive groups, designed to react with the reactive groups in the organopolysiloxane polymer of the external phase (I) in the presence of the catalyst in order to cure the external phase (I), thereby entrapping the internal phase (II) within the body of the water-containing silicone rubber device.

2. A process in accordance with claim 1 characterised in that the reactive groups of the organopolysiloxane polymer having at least two reactive groups per molecule in the external phase are —OH groups and/or condensable groups and the cure package comprises a condensation catalyst and a cross-linker.

3. A process in accordance with claim 1 wherein the reactive groups of the organosiloxane polymer are selected from the group consisting of —Si(OH)₃, —(R^{a})Si(OH)₂, —(R^{a})₂SiOH, —R^{a}Si(OR^{b})₂, —Si(OR^{b})₃, —R^{a}₂SiOR^{b} and —R^{a}₂Si—R^{c}—SiR^{d}ₙ(OR^{b})₃₋ₙ where each R^{a} independently represents a monovalent hydrocarbyl group each R^{b} and R^{d} group is independently selected from the group consisting of alkyl and alkoxy groups in which the alkyl groups have up to 6 carbon atoms; R^{c} is selected from the group consisting of divalent hydrocarbon groups and divalent hydrocarbon groups which are interrupted by one or more siloxane spacers having up to six silicon atoms; and n has the value 0, 1 or 2.

4. A process in accordance with claim 1 characterised in that the condensation catalyst is selected from the group consisting of dialkyltin esters, titanates of the general formula Ti[OR⁵]₄ and zirconates of the general formula Zr[OR⁵]₄, wherein each R⁵ may be the same or different and is selected from the group consisting of monovalent linear hydrocarbon groups, monovalent branched hydrocarbon groups, primary, secondary and tertiary linear aliphatic hydrocarbon groups, primary, secondary and tertiary branched aliphatic hydrocarbon groups, the hydrocarbon groups having from 1 to 10 carbon atoms, chelated titanates, and chelated zirconates.

5. A process in accordance with claim 1 characterised in that the organopolysiloxane polymer in the external phase comprises a free radical polymerisable moiety selected from the group consisting of organopolysiloxane monomers, organopolysiloxane oligomers and organopolysiloxane polymers; and the cure package comprises an organoboron amine catalyst complex.

6. A process in accordance with claim 1 characterised in that the emulsifier is a water-in-silicone emulsifier having a hydrophilic lipophilic balance of less than 5.

7. A process in accordance with claim 1 characterised in that the active ingredient is selected from the group consisting of fragrances, essential oils, preservatives, vitamins and their derivatives, whitening agents, anti-oxidants, ceramides, amino-acid derivatives, polyols, botanical (plant extracts) conditioning agents for skin, sunscreen agents, humectants, emollients, occlusive agents, esters, anti acne agents, antimicrobial agents, antiperspirant agents, deodorant agents, cosmetic biocides, oxidizing agents, reducing agents, skin bleaching agents, skin protectants, cleansing agents, foam boosting agents, insect-repellents, self-tanning agents, pH control agents, antibiotic agents, antiseptic agents, antifungal agents, antibacterial agents, antimicrobial agents, biocides, anti-inflammatory agents, astringents, hormones, anticancer agents, smoking cessation compositions, cardiovascular agents, histamine blockers, bronchodilators, analgesics, anti-arrythmic agents, antihistamines, alpha-I blockers, beta blocker, ACE inhibitor, diuretic agents, anti-aggregants, sedatives, tranquillizers, anticonvulsants, anticoagulant agents, vitamins, anti-aging agents, agents for treating gastric and duodenal ulcers, anti-cellulites, proteolytic enzymes, healing factors, cell growth nutrients and peptides.

8. A process in accordance with claim 1 characterised in that the active ingredient is selected from the group consisting of perfumes, fragrances and essential oils.

9. A process in accordance with claim 1 characterised in that devices prepared by the process of claim 1 are moulded into predetermined shape by curing in a mould to form a soft solid rubber object.

10. (canceled)

11. A process in accordance with claim 1 characterised in that the devices are provided in the form of a water-in-silicone-in-water suspension.

12. (canceled)

13. A device made in accordance with the process of claim 1 having a three dimensional shape, where each dimension has a minimum size of at least 1 millimetre, the device comprising a silicone elastomer having water dispersed in it.

14. (canceled)

15. A device according to claim 13 characterised in that it provides controlled and substantially uniform release of the one or more active ingredients.

16. (canceled)

17. A device according to claim 13, which further comprises silicone and organic diluents of the silicone rubber phase.

18. (canceled)

19. A process in accordance with claim 1 characterised in that the devices are cured to form a flexible rubber thin sheet.
